(19) United States
(12) Patent Application Publication
(10) Pub. No.: US 2023/0381453 A1
(43) Pub. Date: Nov. 30, 2023

(54) CATHETER

(71) Applicant: TEREMO KABUSHIKI KAISHA, Tokyo (JP)

(72) Inventors: Kodai KITAMURA, Kanagawa (JP); Takashi ITO, Shizuoka (JP); Mitsuki HOSHI, Shizuoka (JP)

(73) Assignee: TEREMO KABUSHIKI KAISHA, Tokyo (JP)

(21) Appl. No.: 18/448,248

(22) Filed: Aug. 11, 2023

Related U.S. Application Data
(63) Continuation of application No. PCT/JP2022/007144, filed on Feb. 22, 2022.

(30) Foreign Application Priority Data
Feb. 22, 2021 (JP) 2021-026036

(51) Int. Cl. A61M 25/00

(52) U.S. Cl. CPC A61M 25/005

(57) ABSTRACT

A catheter capable of suppressing separation of an end portion of a reinforcing body while applying a flat wire effective for making the catheter thin and have high strength to the reinforcing body includes a shaft having a lumen communicating from a distal end to a proximal end, in which the shaft includes a reinforcing member including a wire disposed on at least a part between an inner surface of the shaft forming the lumen and an outer surface of the shaft and braided in a tubular shape. The reinforcing body includes a round wire group including plural round wires each of which is a wire having a circular cross-section, and plural flat wires intersecting the round wire group, and a cross-sectional area ratio NB of a total cross-sectional area A of the round wire groups to a total cross-sectional area B of the flat wires is larger than 0.5.

### CROSS-REFERENCES TO RELATED APPLICATIONS

This application is a continuation of International Application No. PCT/JP2022/007144 filed on Feb. 22, 2022, which claims priority to Japanese Patent Application No. 2021-026036 filed on Feb. 22, 2021, the entire content of both of which is incorporated herein by reference.

### TECHNOLOGICAL FIELD

The present invention generally relates to a catheter used in a lumen such as a blood vessel.

### BACKGROUND DISCUSSION

In recent years, intraluminal treatment of a blood vessel or the like using a catheter has been actively performed because surgical invasion is very low. It is desired that the catheter is thin while maintaining strength in a manner that the catheter can be inserted into a thin lumen and a wide passage can be secured inside.

Japanese Patent Application Publication No. 2014-144163 A discloses a method of making a catheter have high strength and thin thickness, a flat wire, which is a strip-shaped wire made of a flat plate, is braided into a tubular shape and embedded in the catheter as a reinforcing body

### SUMMARY

However, since the flat plate has high strength, when the flat wire is braided, the cut end portion of the flat plate is likely to be separated or tend to move outwardly. As a result, there is a possibility that the catheter cannot be easily cut, or when the reinforcing body is embedded in the tubular resin material, the separated reinforcing body cannot come into close contact with the resin material, and the strength of the catheter decreases. For this reason, in general, in a catheter using a flat wire as a reinforcing body, post-processing such as welding wires at end portions of the reinforcing body is required.

The catheter disclosed here is capable of suppressing separation of an end portion of a reinforcing body while applying a flat wire effective for making the catheter thin and have high strength to the reinforcing body.

One aspect of a catheter for achieving the above object is a catheter including a shaft having a lumen communicating from a distal end to a proximal end, in which the shaft includes a reinforcing member including a wire disposed on at least a part between an inner surface of the shaft forming the lumen and an outer surface of the shaft and braided in a tubular shape, the reinforcing body includes a round wire group including a plurality of round wires each of which is a wire having a circular cross-section, and a plurality of flat wires each of which is a wire having a rectangular cross-section and intersecting the round wire group, and a cross-sectional area ratio of a total cross-sectional area of the plurality of round wire groups to a total cross-sectional area of the plurality of flat wires is larger than 0.5.

Another aspect of a catheter for achieving the above object is a catheter comprising a shaft having a lumen communicating from a distal end to a proximal end, in which the shaft includes a reinforcing member including a wire disposed on at least a part between an inner surface of the shaft forming the lumen and an outer surface of the shaft and braided in a tubular shape, the reinforcing body includes a round wire group including a plurality of round wires each of which is a wire having a circular cross-section, and a plurality of flat wires intersecting the round wire group, and a ratio of a diameter of the round wire to a thickness of the flat wire is larger than 1.5.

An additional aspect of a catheter for achieving the above object is a catheter comprising a shaft having a lumen communicating from a distal end to a proximal end, in which the shaft includes a reinforcing member including a plurality of wires disposed on at least a part between an inner surface of the shaft forming the lumen and an outer surface of the shaft and braided in a tubular shape, the reinforcing body includes a plurality of round wires each of which is a wire having a circular cross-section, and a plurality of flat wires intersecting the round wire, and a cross-sectional area ratio of a total cross-sectional area of the plurality of round wires to a total cross-sectional area of the plurality of flat wires is larger than 0.5.

In the catheter configured as described above, it is possible to suppress the end portion of the reinforcing body from being separated by the round wire group which is applied to the reinforcing body together with the flat wire and intersects with the flat wire while applying the flat wire effective for making the catheter thin and high strength to the reinforcing body.

The cross-sectional area ratio may be larger than 1. As a result, the catheter can enhance the effect that the end portion of the reinforcing body is less likely to be separated due to the round wire group.

The yield point of the material of the round wire may be lower than the yield point of the material of the flat wire. As a result, the round wire is more easily plastically deformed than the flat wire, and the braided shape is easily maintained. Therefore, it is possible to effectively suppress the end portion of the reinforcing body from being separated by holding the flat wire which is made to be separated because it is difficult to plastically deform by the round wire group.

The number of the round wires may be larger than the number of the flat wires. As a result, it is possible to effectively suppress the end portion of the reinforcing body from being separated by holding the flat wire which is made to be separated by the round wire.

The intersecting wires may not be joined to each other. As a result, in the catheter, even if the intersecting wires are not joined to each other, it is possible to suppress separation of the reinforcing body. Therefore, it is possible to facilitate processing of the thin catheter with high strength.

The flat wire may be a wire having a rectangular cross-section. As a result, the cross-sectional area of the flat wire increases, and the flat wire can be thinned to obtain the thin catheter with high strength.

In the catheter of still another aspect configured as described above, it is possible to suppress the end portion of the reinforcing body from being separated by the round wire which is applied to the reinforcing body together with the flat wire and intersects with the flat wire while applying the flat wire effective for making the catheter thin and high strength to the reinforcing body.

### BRIEF DESCRIPTION OF THE DRAWINGS

FIG. 1 is a plan view illustrating a catheter according to an embodiment.

FIG. 2 is a longitudinal sectional view along a central axis, illustrating the catheter according to the embodiment.

FIG. 3 is a lateral longitudinal sectional view orthogonal or perpendicular to a central axis, illustrating the catheter according to the embodiment.

FIG. 4 is a plan view illustrating an outer layer of the catheter according to the embodiment in a transparent manner.

FIGS. 5(A) and 5(B) are cross-sectional views illustrating a wire of a reinforcing body, in which FIG. 5(A) illustrates a round wire and FIG. 5(B) illustrates a flat wire.

FIG. 6 is a plan view illustrating, as a reference example, a state in which a wire is separated at an end portion of a cut reinforcing body in a manufacturing process.

### DETAILED DESCRIPTION

Hereinafter, embodiments of the new catheter disclosed here will be described with reference to the accompanying drawings. The dimensions of the drawings may be exaggerated and different from actual dimensions for convenience of description. In the present specification and the drawings, components having substantially the same functional configuration are denoted by the same reference numerals, and a detailed description of such features and aspects will not be repeated. In the present specification, a side to be inserted into a body lumen of a catheter 1 is referred to as a “distal end side”, and a side to be operated is referred to as a “proximal end side”.

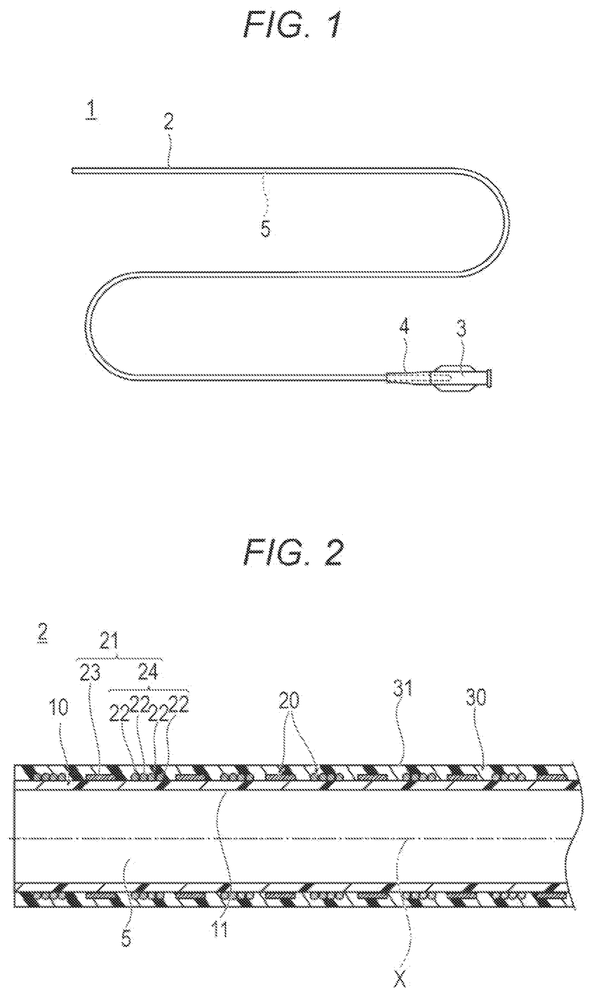

The catheter 1 according to the present embodiment is, during use, introduced into a blood vessel from a radial artery of an arm and inserted into an artery of a lower limb, and is used for performing treatment, diagnosis, or the like. The artery of the lower limb is an artery near the aortoiliac artery bifurcation and on the more peripheral side. As illustrated in FIG. 1, the catheter 1 includes an elongated shaft 2, a hub 3 connected to a proximal end of the shaft 2, and a kink protector 4 provided at a connection portion between the shaft 2 and the hub 3.

As illustrated in FIGS. 1 to 4, the shaft 2 is a tubular member having flexibility, has a central axis X extending from a proximal end to a distal end, and has a lumen 5 formed inside from the proximal end to the distal end. A guide wire is inserted through the lumen 5 when the catheter 1 is inserted into a blood vessel. In addition, the lumen 5 can also be used as a passage for medical instruments such as other catheters 1, medicinal solutions, embolic substances, contrast media, or the like.

The effective length of the shaft 2 is not particularly limited, and is appropriately set according to the application of the catheter 1. In a case where the catheter 1 is introduced into a blood vessel from a radial artery of an arm and inserted into an artery of a lower limb, and is used for performing treatment, diagnosis, or the like, the effective length of the shaft 2 is not particularly limited, but is preferably 1500 mm to 2600 mm, more preferably 1800 mm to 2300 mm, and still more preferably 2100 mm to 2300 mm. As a result, the catheter 1 can reach the artery of the lower limb from the artery of the arm. The effective length of the shaft 2 is a length of a portion that can be inserted into a blood vessel, a sheath, or the like. In the present embodiment, the effective length is a length along the central axis X from the most distal end of the kink protector 4 to the most distal end of the shaft 2. The effective length of the shaft 2 is preferably 650 mm or more in a case where the catheter 1 is introduced into the blood vessel from the femoral artery, and is preferably 300 mm or more in a case where the catheter 1 is introduced into the blood vessel from the distal portion of the dorsalis pedis artery or the posterior tibial artery.

The shaft 2 includes a plurality of layers, and includes an inner layer 10 forming an inner surface 11 of the lumen 5 (inner surface of the shaft), a reinforcing body 20 disposed on the outer side of the inner layer 10, and an outer layer 30 formed on the outer side of the inner layer 10 and the reinforcing body 20.

The lumen 5 is formed inside the inner layer 10. As a constituent material from which the inner layer 10 may be fabricated, a thermoplastic resin, a thermosetting resin, or the like can be applied, and a fluorine-based resin such as polytetrafluoroethylene (PTFE), a low-friction material such as high-density polyethylene (HDPE), or the like is preferable.

The inner diameter of the inner layer 10 is not particularly limited, but is preferably 0.4 mm to 1.2 mm, more preferably 0.45 mm to 0.7 mm, and still more preferably 0.5 mm to 0.6 mm.

The reinforcing body 20 is formed by braiding a plurality of wires 21 in a tubular shape so that a gap exists between axially adjacent portions of the braided wires, and the braided wires are located on the outer periphery of the inner layer 10. The wires 21 include a plurality of round wires 22 having a circular cross-section and a plurality of flat wires 23 having a rectangular cross-section. The cross-section of the wire 21 is a cross-section orthogonal (perpendicular) to the direction in which the wire 21 extends. The cross-section of the flat wire 23 may be a rectangle, an ellipse, or an oval, but in a case where shapes are compared with the same thickness and width, a rectangle having the largest cross-sectional area is preferable.

The round wires 22 form plural round wire groups 24, each with one or a plurality of strands. That is, each round wire group 24 includes one or a plurality of round wires 22 arranged adjacent to each other in the circumferential direction of the shaft 2. Thus, as shown in FIGS. 2-4, the wires 22 in each round wire group 24 are axially adjacent one another and extend helically around the outer periphery of the inner layer 10. The round wire groups 24 each extend in a way that follows a spiral in the same direction around the outer periphery of the inner layer 10, and the windings forming the spiral are disposed at equal intervals in the circumferential direction of the shaft 2. That is, as shown by way of example in FIG. 4, the axial spacing between adjacent windings of the round wire groups 24 The plurality of round wires 22 constituting each round wire group 24 are disposed side by side without a gap in the circumferential direction of the shaft 2. Thus, as shown in FIGS. 2-4, the adjacent wires 22 in each round wire groups 24 contact one another. The plurality of round wires 22 constituting each round wire group 24 may be disposed with some gap in the circumferential direction of the shaft 2, meaning there may be a spacing between adjacent round wires in each round wire group 24. A number L1 of the round wire groups 24 is equal to the number of the flat wires 23, but may not be equal to it. The number L1 of the round wire groups 24 is not particularly limited. If one of the round wire groups 24 is considered one line, the number L1 of round wire groups 24 may be, for example, one (1) line to sixteen (16) lines, and is four (4) lines in the present embodiment. A number N1 of the round wires 22 included in each round wire group 24 is not particularly limited, but is, for example, 1 wire to 8 wires, and is 4 wires in the present embodiment. In a case where a plurality of round wires 22 is included in each round wire group 24, the plurality of round wires 22 having a smaller outer diameter can be used instead of one round wire 22 having a larger outer diameter, thus making it possible to avoid an increase in the wall thickness and the outer diameter of the shaft 2.

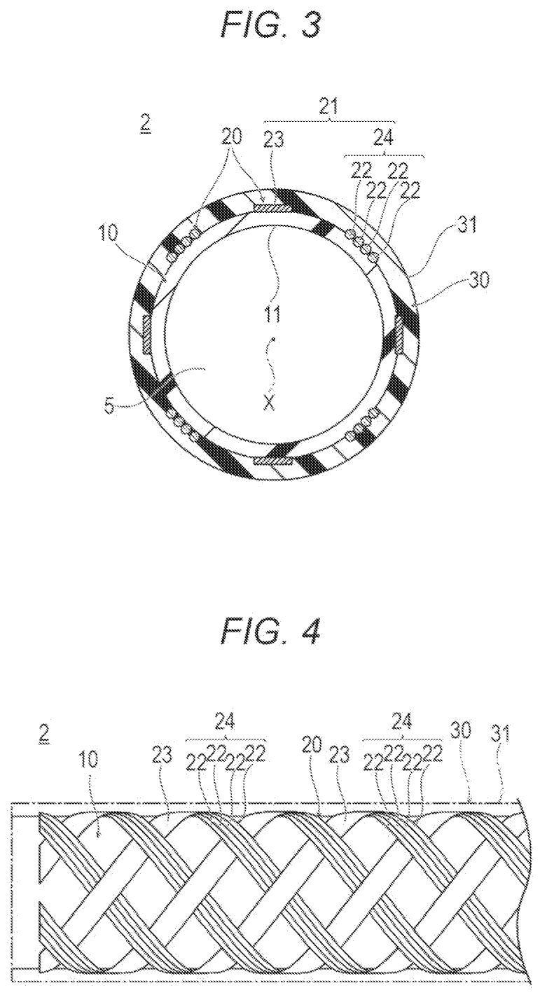

The flat wires 23 extend in the same direction and extend in a manner such that, for each flat wire 23, one piece of a strand (one wire) extends in a spiral manner around the outer periphery of the inner layer 10, and the flat wires 23 are disposed at equal intervals in the circumferential direction of the shaft 2. That is, the axial spacing between adjacent flat wires 23 is the same. The direction in which the plurality of flat wires 23 extend is opposite to the direction in which the round wire groups 24 extend in the circumferential direction of the shaft 2 to intersect with the round wire group 24. The flat wires 23 and the round wire groups 24 are braided in a manner that the arrangement overlapping in the radial direction of the shaft 2 is interchanged alternately or with a pattern. That is, as shown by way of example in FIG. 4, the flat wires 23 and the round wire groups 24 are braided with each other so that they are positioned in alternating overlying and underlying relation to one another. As illustrated in FIG. 3, a number L2 of the flat wires 23 is not particularly limited. If one flat wire is defined as one line, the number L2 of flat wires 23 may be, for example, one line to sixteen lines. The number L2 of the flat wires 23 matches the number L1 of the round wire groups 24, but does not necessarily need to match. Each flat wire 23 is disposed in a manner that the direction of the short side of the rectangular cross-section substantially matches the radial direction of the shaft 2.

As a constituent material from which the round wire 22 and the flat wire 23 may be fabricated, a metal wire such as stainless steel, platinum (Pt), tungsten (W), or the like, a resin fiber, a carbon fiber, a glass fiber, or the like can be applied, or a plurality of these wires 21 may be used in combination.

The constituent materials of the round wire 22 and the flat wire 23 may be the same material, but are preferably different. The yield point of the material of the round wire 22 is preferably lower than the yield point of the material of the flat wire 23. The round wire 22 is deformed beyond the yield point in the braided state. That is, the round wire 22 is plastically deformed when braided, and constitutes the reinforcing body 20 in a state where the restoring force to return to the original shape is smaller than that of the flat wire 23. The flat wire 23 constitutes the reinforcing body 20 in a state where the internal stress remains larger than that of the round wire 22 in the braided state and the restoring force for returning to the original shape is larger than that of the round wire 22.

As an example, the constituent materials of the round wire 22 and the flat wire 23 are different stainless steel, and the yield point of the material of the round wire 22 is lower than the yield point of the material of the flat wire 23. A constituent material of the round wire 22 is, for example, SUS316, and a constituent material of the flat wire 23 is, for example, SUS304-WPB.

In a case where the total (total cross-sectional area) of the cross-sectional areas of all the round wires 22 constituting the reinforcing body 20 is defined as A, and the total (total cross-sectional area) of the cross-sectional areas of all the flat wires 23 constituting the reinforcing body 20 is defined as B, NB is preferably larger than 0.5, more preferably 0.74 or more, more preferably larger than 1, still more preferably 1.4 or more, and still more preferably 2.0 or more. A/B is preferably 2.1 or less, more preferably less than 2.1, and still more preferably 2.09 or less. As a result, it is possible to sufficiently secure the total cross-sectional area of the round wires 22 and improve the effect of maintaining the shape of the cut end portion of the reinforcing body 20 by the plastically deformed round wires 22. The cross-sectional area of each wire 21, 23 is an area in a cross-section orthogonal (perpendicular) to the extending direction of the wires 21, 23.

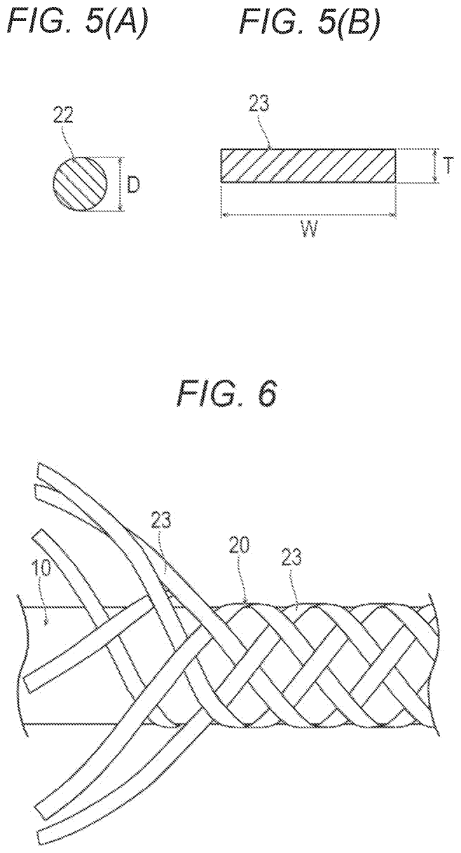

In addition, as illustrated in FIGS. 5(A) and 5(B), in a case where the diameter of the round wire 22 is defined as D and the thickness of the flat wire 23 (the length of the short side of the rectangular cross-section) is defined as T, D/T is preferably larger than 1.5, more preferably 2.0 or more, and still more preferably 4.0 or more. As a result, it is possible to sufficiently secure the diameter of the round wire 22 and improve the effect of maintaining the shape of the cut end portion of the reinforcing body 20 by the plastically deformed round wire 22. A diameter D of the round wire 22 is a diameter in a cross-section orthogonal to the extending direction of the round wire 22. A thickness T of the flat wire 23 is the length of the short side of the rectangular cross-section in the cross-section orthogonal to the extending direction of the flat wire 23. A width W of the flat wire 23 is the length of the long side of the rectangular cross-section in the cross-section orthogonal to the extending direction of the flat wire 23.

The diameter D of the round wire 22 is not particularly limited, but is preferably 0.020 mm to 0.080 mm, more preferably 0.025 mm to 0.060 mm, and still more preferably 0.030 mm to 0.040 mm.

The thickness T of the flat wire 23 is not particularly limited, but is preferably 0.010 mm to 0.040 mm, more preferably 0.010 mm to 0.030 mm, and still more preferably 0.015 mm to 0.020 mm. The width W of the flat wire 23 is not particularly limited, but is preferably 0.045 mm to 0.250 mm, more preferably 0.045 mm to 0.200 mm, and still more preferably 0.045 mm to 0.140 mm.

As illustrated in FIGS. 2 to 4, the outer layer 30 is a tubular member that covers the outer periphery of the inner layer 10 and the reinforcing body 20. The outer layer 30 forms an outer surface 31 which is a radially outer surface of the shaft 2.

The outer diameter of the outer layer 30 is not particularly limited, but is preferably 0.7 mm to 1.3 mm, more preferably 0.8 mm to 1.2 mm, and still more preferably 0.86 mm to 1.1 mm.

Examples of a constituent material from which the outer layer 30 may be fabricated include a polymer material such as polyolefin (for example, polyethylene, polypropylene, polybutene, an ethylene-propylene copolymer, an ethylene-vinyl acetate copolymer, an ionomer, or a mixture of two or more kinds of these), polyvinyl chloride, polyamide, a polyester elastomer, a polyamide elastomer, polyurethane, a polyurethane elastomer, polyimide, or a fluororesin, a thermoplastic resin such as a mixture of these, or a thermosetting resin such as an epoxy resin. The radiopaque substance may be mixed with the outer layer 30.

As illustrated in FIG. 1, in the hub 3, a proximal end portion of the shaft 2 is liquid-tightly fixed by an adhesive, heat fusion, a stopper (not illustrated), or the like. The hub 3 functions as a guide wire or an insertion port of a medical instrument into the lumen 5, an injection port of a medicinal solution, an embolic substance, a contrast medium, or the like into the lumen 5, and also functions as a grip portion when the catheter 1 is operated. The constituent material from which the hub 3 may be fabricated is not particularly limited, but for example, thermoplastic resins such as polycarbonate, polyamide, polysulfone, polyarylate, and a methacrylate-butylene-styrene copolymer can be suitably used.

The kink protector 4 is made of an elastic material provided to surround the periphery of the shaft 2, and suppresses kink of the shaft 2 at a connection portion between the shaft 2 and the hub 3. Examples of a constituent material from which the kink protector 4 may be fabricated include natural rubber, silicone resin, polyamide elastomer, polyester elastomer, or the like.

Next, a method for manufacturing the catheter 1 according to the present embodiment will be described.

First, a long or elongated core wire having an outer diameter equal to the inner diameter of the inner layer 10 is prepared. Next, the inner layer 10 is formed on the core wire. The inner layer 10 may be formed by extrusion or by dip forming. Alternatively, the core wire may be inserted into the lumen 5 of the inner layer 10 which is a tubular body.

Thereafter, as shown in FIG. 4, the reinforcing body 20 is formed to cover at least a part of the inner layer 10. The reinforcing body 20 is formed by continuously winding a plurality of round wires 22 and flat wires 23 on the inner layer 10 using a braiding machine (braider). The flat wire 23 can impart high strength to the shaft 2 while maintaining the formed shaft 2 thin (i.e., while maintain a thin configuration of the formed shaft 2). The flat wire 23 constitutes the reinforcing body 20 in a state where the internal stress of the flat wire 23 remains larger than that of the round wire 22 in the braided state and the restoring force for returning the flat wire to the original shape is larger than that of the round wire 22. The round wire 22 is deformed beyond the yield point in the braided state. That is, the round wire 22 is plastically deformed when braided (the round wire 22 becomes plastically deformed as a result of braiding), and constitutes the reinforcing body 20 in a stable state where the restoring force to return the round wire 22 to the original shape is smaller than that of the flat wire 23.

Next, the end portion of the reinforcing body 20 is cut. At this time, the cut end portion of the flat wires 23 having high strength and a high yield point is likely to be spread and separated by its own restoring force as in the reference example of FIG. 6. Here, the separation means that the tubular wound reinforcing wires (for example, the wires 21) spreads to five times or more the outer diameter in a natural state. However, in the present embodiment, since the flat wires 23 intersect with the round wires 22 which are plastically deformed and have small and stable internal stress, the shape of the end portion of the cut flat wires 23 is held by the round wires 22. Therefore, the reinforcing body 20 can suppress the separation of the wires 21 (the round wires 22 and the flat wires 23) at the cut end portion.

Next, the reinforcing body 20 is pressed from the outer peripheral surface side, and the inner peripheral surface side of the reinforcing body 20 is embedded in the inner layer 10. Thereafter, as shown in FIGS. 2 to 3, the outer layer 30 is formed on the outer side of the inner layer 10 and the reinforcing body 20. A method for forming the outer layer 30 is not particularly limited. For example, the outer layer 30 may be formed by extrusion molding or dip molding. Alternatively, the outer layer 30 may be formed by disposing a tubular body as a material of the outer layer 30 on the outer side of the inner layer 10 and the reinforcing body 20, and then covering the tubular body with a heat shrinkable tube and heating the tubular body. While being softened or melted by heating, the tubular body is joined in close contact with the outer side of the inner layer 10 and the reinforcing body 20 by the contraction force of the heat shrinkable tube. After this, the heat shrinkable tube is removed.

After forming the outer layer 30, the core wire is pulled out from the lumen 5 of the inner layer 10. After this, the hub 3 and the kink protector 4 are attached to the shaft 2, and other members (for example, the distal end tip) are attached as necessary to complete the catheter 1. Alternatively, the distal end tip may be attached from the beginning and molded.

As described above, the catheter 1 according to the present embodiment is the catheter 1 including the shaft 2 having the lumen 5 communicating from the distal end to the proximal end, in which the shaft 2 has the reinforcing body 20 including the wire 21 disposed at least partially between the inner surface 11 of the shaft 2 forming the lumen 5 and the outer surface 31 of the shaft 2 and braided in a tubular shape, the reinforcing body 20 has the round wire group 24 including the plurality of round wires 22 which are the wires 21 having a circular cross-section and the plurality of flat wires 23 which are the wires 21 having a rectangular cross-section and intersecting the round wire group 24, and the cross-sectional area ratio A/B of the total cross-sectional area A of the plurality of round wire groups 24 to the total cross-sectional area B of the plurality of flat wires 23 is larger than 0.5.

Alternatively, the catheter 1 according to the present embodiment is a catheter 1 catheter including a shaft 2 having a lumen 5 communicating from a distal end to a proximal end, in which the shaft 2 includes a reinforcing body 20 including a wire 21 disposed on at least a part between an inner surface 11 of the shaft 2 forming the lumen 5 and an outer surface 31 of the shaft 2 and braided in a tubular shape, the reinforcing body 20 includes a round wire group 24 including a plurality of round wires 22 each of which is a wire 21 having a circular cross-section, and a plurality of flat wires 23 each of which is a wire 21 having a rectangular cross-section and intersecting the round wire group 24, and a ratio D/T of a diameter D of the round wire 22 to a thickness T of the flat wire 23 is larger than 1.5.

In the catheter 1 configured as described above, it is possible to suppress the end portion of the reinforcing body 20 from being separated by the round wire group 24 which is applied to the reinforcing body 20 together with the flat wire 23 and intersects with the flat wire 23 while applying the flat wire 23 effective for making the catheter 1 thin and high strength to the reinforcing body 20. Therefore, it is possible to facilitate processing of the thin catheter 1 with high strength. Here, the separation means that the maximum outer diameter of the end portion of the reinforcing body 20 when the reinforcement wire (wire of the reinforcing body 20) is cut is five times or more the outer diameter of the intermediate portion of the reinforcing body 20 that is not separated.

In addition, the cross-sectional area ratio NB may be larger than 1. As a result, it is possible to enhance the effect that the end portion of the reinforcing body 20 is less likely to be separated due to the round wire group 24.

In addition, the yield point of the material of the round wire 22 is lower than the yield point of the material of the flat wire 23. As a result, the round wire 22 is more easily plastically deformed than the flat wire 23, and the braided shape is easily maintained. Therefore, it is possible to effectively suppress the end portion of the reinforcing body 20 from being separated by holding the flat wire 23 which is made to be separated because it is difficult to plastically deform by the round wire group 24.

In addition, the number of round wires 22 is larger than the number of flat wires 23. As a result, it is possible to effectively suppress the end portion of the reinforcing body 20 from being separated by holding the flat wire 23 which is made to be separated by the round wire 22.

In addition, the intersecting wires 21 are not joined to each other by welding, bonding, or the like. In the catheter 1, even if the intersecting wires 21 are not joined to each other, it is possible to suppress separation of the reinforcing body 20. Therefore, it is possible to facilitate processing of the thin catheter 1 with high strength.

In addition, the effective length of the shaft 2 is 2100 mm or more. As a result, the catheter 1 can easily reach the artery of the lower limb from the artery of the arm.

In addition, the flat wire 23 is a wire having a rectangular cross-section. As a result, the cross-sectional area of the flat wire 23 increases, and the flat wire 23 can be thinned (the thickness of the wire 23 can be reduced) to obtain the thin catheter 1 with high strength.

As a modification, strands of the round wire 22 and the flat wire 23 may be braided. In the braided catheter configured as described above, it is possible to suppress the end portion of the reinforcing body 20 from being separated by the round wire 22 which is applied to the reinforcing body 20 together with the flat wire 23 and intersects with the flat wire 23 while applying the flat wire 23 effective for making the catheter thin and high strength to the reinforcing body 20.

### EXAMPLES

Hereinafter, examples of catheters embodying aspects of the disclosure here and comparative examples will be described. The present invention is not limited to these examples.

The shaft 2 of Examples 1 to 5 and Comparative Example 1 shown in Table 1 has been produced, and whether or not the cut end portion of the reinforcing body 20 becomes separated has been observed in the process of producing the shaft 2. In all of Examples 1 to 5 and Comparative Example 1, the material of the round wire 22 is SUS316 and the material of the flat wire 23 is SUS304-WPB.

In Example 1, the disposition of the round wires 22 has been braided by setting the braiding machine in a manner that the number L1 of the round wire groups 24 is 8 lines, and the number N1 of the round wires 22 constituting each round wire group 24 is 2 wires, but after the braiding, the two adjacent round wire groups 24 have been combined, and substantially, the number L1 of the round wire groups 24 has been 4 lines, and the number N1 of the strands of the round wires 22 constituting each round wire group 24 has been 4 wires.

As a result, in Examples 1 to 5 in which the cross-sectional area ratio NB is larger than 0.5 and smaller than 2.1, the result that the cut end portions of the reinforcing body 20 are not separated (or hardly separated) has been obtained, as compared with Comparative Example 1 in which the cross-sectional area ratio A/B is smaller than 0.5. In addition, in Examples 1 to 5 in which a thickness ratio D/T is larger than 1.5, the result that the cut end portions of the reinforcing body 20 are not separated (or hardly separated) has been obtained, as compared with Comparative Example 1 in which the thickness ratio D/T is smaller than 1.5.

TABLE 1

| | Round wire | | | | Flat wire | | | | | | | |
|---|---|---|---|---|---|---|---|---|---|---|---|---|
| | | | | Total | | | | | Total | Cross- | | |
| | | | | cross- | | | | | cross- | sec- | | |
| | Wire | Wire | | sec- | Wire | Thick- | | | sec- | tional | Thick- | |
| | compo- | diam- | Number | tional | compo- | ness | Width | Number | tional | area | ness | |
| | sition | eter D | of | area A | sition | T | W | of | area B | ratio | ratio | |
| | N1 × L1 | [mm] | wires | [mm²] | L2 | [mm] | [mm] | wires | [mm²] | A/B | D/T | Result |
| Example | 2 wires × | 0.04 | 16 | 0.020 | 4 lines | 0.02 | 0.17 | 4 | 0.014 | 1.48 | 2.00 | Not |
| 1 | 8 lines | | | | | | | | | | | separated |
| Example | 2 wires × | 0.04 | 8 | 0.010 | 4 lines | 0.02 | 0.17 | 4 | 0.014 | 0.74 | 2.00 | Not |
| 2 | 4 lines | | | | | | | | | | | separated |
| Example | 1 wire × | 0.08 | 4 | 0.020 | 4 lines | 0.02 | 0.17 | 4 | 0.014 | 1.48 | 4.00 | Not |
| 3 | 4 lines | | | | | | | | | | | separated |
| Com- | 2 wires × | 0.03 | 8 | 0.006 | 4 lines | 0.02 | 0.17 | 4 | 0.014 | 0.42 | 1.50 | Becomes |
| parative | 4 lines | | | | | | | | | | | separated |
| Example | | | | | | | | | | | | |
| 1 | | | | | | | | | | | | |
| Example | 2 wires × | 0.03 | 8 | 0.006 | 4 lines | 0.015 | 0.045 | 4 | 0.003 | 2.09 | 2.00 | Not |
| 4 | 4 lines | | | | | | | | | | | separated |
| Example | 2 wires × | 0.03 | 16 | 0.011 | 8 lines | 0.01 | 0.08 | 8 | 0.006 | 1.76 | 3.00 | Not |
| 5 | 8 lines | | | | | | | | | | | separated |

The invention is not limited to the above embodiments, and various modifications may be made within the technical idea of the invention by those skilled in the art. For example, the catheter 1 may be inserted from a blood vessel other than the artery of the arm. In addition, the catheter 1 may be used for treatment or diagnosis of a blood vessel other than an artery of a lower limb. In addition, the catheter 1 may be inserted into a bile duct, a trachea, an esophagus, a urethra, or other body lumen or body cavity to be used for treatment, diagnosis, or the like.

The detailed description above describes and embodiment and modification possibilities of a catheter representing examples of the new catheter disclosed here. The invention is not limited, however, to the precise embodiments and modification possibilities described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

1 Catheter
2 Shaft
5 Lumen
10 Inner layer
11 Inner surface
20 Reinforcing body
21 Wire
22 Round wire
23 Flat wire
24 Round wire group
30 Outer layer
31 Outer surface
A Total cross-sectional area of round wire
B Total cross-sectional area of flat wire
D Diameter of round wire
T Thickness of flat wire
W Width of flat wire

What is claimed is:

1. A catheter comprising:
a shaft possessing proximal and distal ends, the shaft having a lumen extending from the distal end of the shaft to the proximal end of the shaft, the shaft possessing an inner surface surrounding the lumen and an outer surface;
the shaft including a reinforcing member disposed on at least a part between the inner surface of the shaft and the outer surface of the shaft;
the reinforcing member including plural wires that are braided in a tubular shape;
the plural wires comprising the reinforcing body including a round wire group of wires and a plurality of flat wires, the round wire group of wires being comprised of a plurality of round wires each of which is a wire having a circular cross-section, the plurality of flat wires intersecting the round wire group of wires; and
a cross-sectional area ratio of a total cross-sectional area of the plurality of wires comprising the round wire group of wires to a total cross-sectional area of the plurality of flat wires is larger than 0.5.

2. The catheter according to claim 1, wherein the cross-sectional area ratio is larger than 1.

3. The catheter according to claim 1, wherein all of the plurality of wires comprising the round wire group of wires are in contact with one another.

4. The catheter according to claim 1, wherein the round wire group of wires and the plurality of flat wires are positioned so that in a cross-section of the shaft perpendicular to a central axis of the shaft, the plurality of wires comprising the round wire group of wires are positioned between two of the flat wires.

5. The catheter according to claim 1, wherein the reinforcing body includes a plurality of round wire groups of wires each of which includes a plurality of round wires.

6. The catheter according to claim 5, wherein the round wire groups of wires and the plurality of flat wires are positioned so that in a cross-section of the shaft perpendicular to a central axis of the shaft, the round wire group of wires and the flat wires alternate with one another in a circumferential direction.

7. The catheter according to claim 1, wherein each of the round wires in the round wire group of wires is made of a material having a yield point that is lower than a yield point of a material from which the flat wire is made.

8. The catheter according to claim 1, wherein each of the round wires in the round wire group of wires is plastically deformed.

9. A catheter comprising:
a shaft possessing proximal and distal ends, the shaft having a lumen extending from the distal end of the shaft to the proximal end of the shaft, the shaft possessing an inner surface surrounding the lumen and an outer surface;
the shaft including a reinforcing member disposed on at least a part between the inner surface of the shaft and the outer surface of the shaft;
the reinforcing member including plural wires that are braided in a tubular shape;
the plural wires comprising the reinforcing body including a round wire group of wires and a plurality of flat wires, the round wire group of wires being comprised of a plurality of round wires each of which is a wire having a circular cross-section, the plurality of flat wires intersecting the round wire group of wires, each of the plurality of round wires possessing an outer diameter and each of the plurality of flat wires possessing a thickness; and
a ratio of the outer diameter of the round wires to the thickness of the flat wires is larger than 1.5.

10. The catheter according to claim 9, wherein a yield point of a material from which the round wires are made is lower than a yield point of a material from which the flat wire is made.

11. The catheter according to claim 9, wherein the reinforcing member includes a plurality of round wire groups of wires, each of the plurality of round wire groups of wires being comprised of a plurality of round wires each having a circular cross-section, a total number of the round wires in the reinforcing member being larger than a total number of the flat wires in the reinforcing member.

12. The catheter according to claim 9, wherein the plurality of flat wires intersecting the round wire group of wires are not joined to each other.

13. The catheter according to claim 9, wherein the flat wire is a wire having a rectangular cross-section.

14. The catheter according to claim 9, wherein all of the plurality of wires comprising the round wire group of wires are in contact with one another.

15. The catheter according to claim 9, wherein the round wire group of wires and the plurality of flat wires are positioned so that in a cross-section of the shaft perpendicular to a central axis of the shaft, the plurality of wires comprising the round wire group of wires are positioned between two of the flat wires.

16. The catheter according to claim 9, wherein each of the round wires in the round wire group of wires is plastically deformed.

17. A catheter comprising:
a shaft possessing proximal and distal ends, the shaft having a lumen extending from the distal end of the shaft to the proximal end of the shaft, the shaft possessing an inner surface surrounding the lumen and an outer surface;
the shaft including a reinforcing member disposed on at least a part between the inner surface of the shaft and the outer surface of the shaft;
the reinforcing member including plural wires that are braided in a tubular shape;
the plural wires comprising the reinforcing body including a plurality of round wires each of which is a wire having a circular cross-section, and a plurality of flat wires intersecting the round wires;
a cross-sectional area ratio of a total cross-sectional area of the plurality of round wires to a total cross-sectional area of the plurality of flat wires is larger than 0.5.

18. The catheter according to claim 17, wherein each of the round wires is plastically deformed.

19. The catheter according to claim 17, wherein the plurality of round wires and the plurality of flat wires are positioned so that in a cross-section of the shaft perpendicular to a central axis of the shaft, a plurality of the round wires are positioned between each closest pair of flat wires.

20. The catheter according to claim 17, wherein the plurality of round wires and the plurality of flat wires extend helically along the shaft.
